(19) United States
(12) Patent Application Publication
(10) Pub. No.: US 2007/0292535 A1
(43) Pub. Date: Dec. 20, 2007

(54) Strontium compositions and methods of treating arthritic and or osteoporitic conditions

(76) Inventor: Philip S. Tabbiner, Wilmington, NC (US)

Correspondence Address: HUTCHISON LAW GROUP PLLC, PO BOX 31686, RALEIGH, NC, 27612, US

(21) Appl. No.: 11/455,585

(22) Filed: Jun. 19, 2006

(51) Int. Cl. A61K 33/06; A61K 31/385; A61K 31/355; A61K 31/195

(52) U.S. Cl. 424/682; 514/440; 514/567; 514/458

(57) ABSTRACT

A therapeutic composition and method for treating symptoms or etiology of arthritic conditions by administering a divalent cationic source of strontium, para-aminobenzoic acid and α-lipoic acid is described. In addition, a therapeutic composition and method for treating symptoms or etiology of arthritic conditions by administering a divalent cationic source of strontium, para-aminobenzoic acid, α-lipoic acid and vitamin E is described. Further, a therapeutic composition and method for treating symptoms or etiology of osteoporitic conditions by administering a divalent cationic source of strontium, para-aminobenzoic acid, vitamin B₆, vitamin B₁₂ and folic acid or folate is described.

### FIELD OF THE INVENTION

This invention relates to compositions for and therapeutic treatments of the symptoms and etiology of arthritic conditions and osteoporitic conditions.

### BACKGROUND

Arthritis is a general term for over 100 conditions that affect the joints and surrounding tissues. The two most common types of arthritis are osteoarthritis and rheumatoid arthritis.

Osteoarthritis is a painful, degenerative joint disease that often involves the hips, knees, neck, lower back, or the small joints of the hands. Osteoarthritis usually develops in joints that are injured by repeated overuse in the performance of a particular job or a favorite sport or from carrying around excess body weight. Eventually this injury or repeated impact thins or wears away the cartilage that cushions the ends of the bones in the joint so that the bones rub together, causing a grating sensation. Joint flexibility is reduced, bony spurs develop, and the joint swells. Usually, the first symptom a person has with osteoarthritis is pain that worsens following exercise or immobility.

Rheumatoid arthritis is an autoimmune inflammatory disease in which the body releases enzymes that attack its own healthy tissues. In rheumatoid arthritis, these enzymes destroy the linings of joints causing pain, swelling, stiffness, deformity, and reduced movement and function. Rheumatoid arthritis also may include systemic symptoms. There is no cure for osteoarthritis or rheumatoid arthritis, however, several drugs and medication options are approved for the prevention and treatment of these conditions.

In addition to osteoarthritis and rheumatoid arthritis, osteoporosis is a disease of worldwide concern in which bones become fragile and more likely to break. If not prevented or if left untreated, osteoporosis can progress painlessly until a bone breaks. These broken bones, also known as fractures, occur typically in the hip, spine, and wrist. Any bone can be affected, but of special concern are fractures of the hip and spine. A hip fracture almost always requires hospitalization and major surgery. It can impair a person's ability to walk unassisted and may cause prolonged or permanent disability or even death. Spinal or vertebral fractures also have serious consequences, including loss of height, severe back pain, and deformity.

Osteoporitic fractures can lead to severely reduced quality of life and morbidity burden, and in some cases to premature mortality. Osteoporosis is a major health threat for 44 million Americans, 68% of whom are women. At least one half of adult women and 1 in 5 adult men over the age of 50, will sustain one or more vertebral, hip or other fractures. The annual social care and acute costs for treating osteoporosis related injuries is estimated to be over 10 billion dollars and is expected to increase. Osteoporosis is diagnosed by a bone mineral density (BMD) test, which is a qualitative way to detect low bone density. There is no cure for osteoporosis, however, several drugs and medication options are approved for the prevention and treatment of osteoporosis.

While it is possible to have both arthritis and osteoporosis, people with osteoarthritis may be less likely to develop osteoporosis. On the other hand, people with rheumatoid arthritis may be more likely to develop osteoporosis, especially as a secondary condition from drugs used in rheumatoid arthritis treatment. Therefore, there is a need to continually develop treatments and regimens for the treatment of these conditions.

### SUMMARY

In one embodiment, a therapeutic dosage form is provided for treating an arthritic condition. The therapeutic dosage form comprises a divalent cationic source of strontium in an amount of from about 50 milligrams to about 400 milligrams, para-aminobenzoic acid and α-lipoic acid.

In another embodiment, a therapeutic dosage form is provided for treating an arthritic condition. The therapeutic dosage form comprises a divalent cationic source of strontium in an amount of from about 50 milligrams to about 400 milligrams, para-aminobenzoic acid, α-lipoic acid and vitamin E.

In one embodiment, a method for treating symptoms or etiology of an arthritic condition in a human is provided. The method comprises administering to a human in need thereof a therapeutically effective amount of a therapeutic dosage form, the therapeutic dosage form comprising a divalent cationic source of strontium, para-aminobenzoic acid and α-lipoic acid.

In another embodiment, a method for treating symptoms or etiology of an arthritic condition in a human is provided. The method comprises administering to a human in need thereof a therapeutically effective amount of a therapeutic dosage form, the therapeutic dosage form comprising a divalent cationic source of strontium, para-aminobenzoic acid, α-lipoic acid and vitamin E.

In one embodiment, a therapeutic dosage form is provided comprising a divalent cationic source of strontium of at least about 50 milligrams, para-aminobenzoic acid, vitamin B₆, vitamin B₁₂ and folic acid or folate.

In one embodiment, a method is provided for treating symptoms or etiology of osteoporosis in a subject. The method comprises the step of administering to a mammal in need thereof a therapeutically effective amount of a therapeutic dosage form comprising a divalent cationic source of strontium, para-aminobenzoic acid, vitamin B₆, vitamin B₁₂ and folic acid or folate.

Use of the combination of substances by the present method may facilitate an increase in mobility of the joints, a reduction of the levels of pain and/or increase in bone mass. The forgoing and other features, advantages and embodiments of the present invention may be more fully appreciated by reference to the following detailed description.

### DETAILED DESCRIPTION OF THE EMBODIMENTS

As used herein, “pharmacologically active agent” or “active agent” are used interchangeably and refer to a compound or composition of matter which, when administered to an organism (human or animal) induces a desired pharmacologic and/or physiologic effect by local and/or systemic action.

As used herein, “therapeutically effective amount” refers to an amount of an active agent that is nontoxic but sufficient to provide the desired effect. For example, a therapeutically effective amount of a divalent cationic source of strontium is an amount sufficient to measurably decrease the symptom or etiology of an arthritic condition. The therapeutically effective amount varies according to the patient's sex, age and weight, the route of administration, the nature of the condition and any treatments which may be associated therewith, or any concurrent related or unrelated treatments or conditions of the patient. Therapeutically effective amounts can be determined without undue experimentation by any person skilled in the art or by following the exemplary guidelines set forth in this application.

As used herein, “pharmaceutical dosage form” refers to a dosage form of an active agent (e.g., tablet, film, injectable, powder, capsule, and the like) which is generally safe, non-toxic and neither biologically nor otherwise undesirable. A pharmaceutical dosage form includes that which is acceptable for veterinary use as well as human pharmaceutical use, and which possesses the necessary and desirable characteristics of a dosage form acceptable for administration to a patient (e.g., a tablet of acceptable hardness, dissolution, stability, and a size and weight practical for oral administration). A pharmaceutical dosage form may include multiple tablets or capsules each of which comprises some part or fraction of the active agents for patient administration such that the multiple tablets or capsules taken together comprise the pharmaceutical dosage form.

As used herein, arthritic condition refers to symptoms and etiology of an osteoarthritic condition, rheumatoid arthritic condition, juvenile chronic arthritis associated condition, juvenile idiopathic arthritis associated condition, Spondyloarthropathies (such as ankylosing spondylitis (Mb Bechterew) and reactive arthritis (Reiter's syndrome)) associated condition, condition associated with psoriatic arthritis, gout condition, condition associated with pseudogout (pyrophosphate arthritis), condition associated with systemic lupus erythematosus (SLE), condition associated with systemic sclerosis (scleroderma), condition associated with Behcet's disease, condition associated with relapsing polychondritis, condition associated with adult Still's disease, condition associated with transient regional osteoporosis, condition associated with neuropathic arthropathy, condition associated with sarcoidosis, arthritic condition, rheumatic condition, joint condition, osteoarthritis joint condition, rheumatoid arthritic joint condition, juvenile chronic arthritis associated joint condition, juvenile idiopathic arthritis associated joint condition, Spondyloarthropathies (such as ankylosing spondylitis (Mb Bechterew) and reactive arthritis (Reiter's syndrome)) associated joint condition, joint condition associated with psoriatic arthritis, gout joint condition, joint condition associated with pseudogout (pyrophosphate arthritis), joint condition associated with systemic lupus erythematosus (SLE), joint condition associated with systemic sclerosis (scleroderma), joint condition associated with Behcet's disease, joint condition associated with relapsing polychondritis, joint condition associated with adult Still's disease, joint condition associated with transient regional osteoporosis, joint condition associated with neuropathic arthropathy, joint condition associated with sarcoidosis, arthritic joint condition, rheumatic joint condition, acute condition, acute joint condition, chronic condition, chronic joint condition, inflammatory condition, inflammatory joint condition, mechanical condition, mechanical joint condition, condition associated with the fibromyalgia syndrome (EMS), condition associated with polymyalgia rheumatics, monarticular joint condition, polyarticular joint condition, nociceptiv condition, neuropathic condition, psychogenous condition, condition of unknown etiology, and conditions mediated by IL-6, IL-6 soluble receptor, or IL-6 receptor.

As used herein, osteoporitic condition refers to symptoms and etiology of osteoporosis.

The term “divalent cationic source of strontium” refers generally to salts of strontium and specifically to when strontium is a divalent cation, and is therefore independent of the nature of the anion. By way of example, 650 milligrams of strontium gluconate is approximately 119 milligrams of a divalent cationic source of strontium (e.g., atomic weight of strontium÷formula weight of strontium salt)×(weight of strontium salt)=weight of divalent cationic source of strontium). Divalent cationic sources of strontium may include distrontium salts. Strontium and distrontium salts may include any and all hydrates thereof and mixtures of hydrates. By way of example, a source of divalent cationic strontium may be strontium acetyl salicylate, strontium acetyloxy-benzoate, strontium ascorbate, strontium aspartate in either L and/or D-form, strontium benzenesulfonate, strontium butyrate, strontium camphorate, strontium carbonate, strontium clodronate, strontium chloride, strontium citrate, strontium ethanesulfonate, strontium fumarate, strontium gluconate, strontium glutamate in either L- and/or D-form, strontium glutarate, strontium ibandronate, strontium ibuprofenate, strontium ketoprofenate, strontium lactate, strontium L-threonate, strontium malate, strontium maleate, strontium maleate, strontium malonate, strontium methanesulfonate, strontium naproxenate, strontium nitrate, strontium oxalate, strontium phosphate, strontium pyruvate, strontium ranelate, strontium risedronate, strontium salicylate, strontium succinate, strontium sulfate, strontium tartrate and mixtures thereof.

As used herein, para-aminobenzoic acid and PABA are used interchangeably and includes organic, transition metal, alkali metal, and alkali earth metal salts thereof.

As used herein, α-lipoic acid is synonymous with 1,2-dithiolane-3-pentanoic acid, 6,8-dithiooctanoic acid and 6,8-thioctic acid, including the D and L isomers and racemates thereof, and includes naturally or synthetically prepared material.

As used herein, vitamin E refers to naturally or synthetically prepared α-tocopherol and all tocol and tocotrienol derivatives exhibiting qualitatively the biological activity of α-tocopherol including derivatives, salts and mixtures thereof.

As used herein, vitamin B₆ refers to pyridoxine HCl, pyridoxal, pyridoxal HCl, pyridoxal 5-phosphate, pyridoxal 5-phosphate calcium salt, pyridoxamine, pyridoxamine dihydrochloride, pyridoxamine phosphate and mixtures thereof.

As used herein, “vitamin B₁₂” refers to cobalamins comprising a 5,6-dimethylbenzimidazole heterocyclic base and derivatives, analogs and coenzymatically active forms thereof. For example, vitamin B₁₂ includes cobalamin, cobamide, cyanocobalamin, aquacobalamin, hydroxocobalamin, co-methylcobalamin and nitritocobalamin and mixtures thereof.

As used herein, folic acid or folates are synonymous with pteroylglutamic acid and pteroylglutamate, respectively. The term folates includes any member of the family of pteroylglutamates, (D or L isomers and racemates) or mixtures of them, having various levels of reduction of the pteridine ring, one-carbon substitutions and substitutions on the glutamate residues and mixtures thereof.

As used herein, tablet or capsules refers generally to solid, gelatinous dosage forms containing active agents with or without suitable diluents and prepared either by compression or molding methods known in the art. Tablets may be discoid in shape, or they may also be round, oval, oblong, cylindrical, or triangular. Tablets may include buccal forms, sublingual forms, oral disintegrating forms and oral care strips. They may differ in size and weight depending on the amount of active agents present and the intended method of administration. The tablets may be compressed tablets, molded tablets or tablet triturates. Tablets may include coated tablets, sugar-coated tablets, buccal tablets, oral disintegrating tablets, and sublingual tablets, or in other forms. Buccal drug delivery, e.g., delivery of a drug by passage of a drug through the buccal mucosa into the bloodstream, may be effected by placing a buccal dosage form on the upper gum or opposing inner lip area of the subject. For buccal administration tablets or lozenges formulated in the conventional manner may be used. Penetration enhancers or permeation enhancers to an increase the permeability of the buccal mucosal tissue to a pharmacologically active agent, e.g., so that the rate at which the drug permeates through the mucosal tissue is increased, may be included therein.

Subjects who may have difficulty swallowing a large tablet, due to esophageal strictures or other pathology, for example, may be administered a therapeutically effective solution of the active agents herein disclosed via a suspension in a pharmaceutically acceptable carrier. Alternatively, such subjects may be provided a liquid, buccal or sublingual form or an oral care strip to be introduced to the oral mucosa.

In addition to the pharmacologically active agent, pharmaceutical dosage forms may contain a number of inert materials or additives. Inert materials and additives may include materials that help in the manufacture of the tablet or to impart satisfactory compression characteristics to the formulation. Inert materials and additives may also include materials that help to give additional desirable physical characteristics to the finished tablet, such as colors, flavors, and sweetening agents. Such inert materials and additives should not materially affect the pharmacological properties of the active agent or agents.

Pharmaceutical dosage forms, e.g., tablets or capsules, may contain one or more excipients or vehicles chosen from diluents, lubricants, binders, disintegrating agents, absorbents, and the like. By way of example and without limitation the diluents may include lactose, dextrose, sucrose, mannitol, sorbitol, cellulose, and/or glycerin. The lubricants may include silica, talc, stearic acid and its magnesium and calcium salts, and/or polyethyleneglycol. The binders may include aluminum and magnesium silicate, starch, gelatin, tragacanth, methyl-cellulose, sodium carboxymethylcellulose and/or polyvinylpyrrolidone.

The dissolution rate of a pharmaceutical dosage form, e.g., tablet or capsule, may be increased by the addition of disintegrant or solubilizing substances, such as, for example, alginic acid, amylose croscarmellose sodium, calcium alginate, calcium carbonate, calcium phosphate, carboxymethylcellulose, carboxymethylcellulose calcium, crospovidone, formaldehyde gelatine, lowly-substituted hydroxypropylcellulose, magnesium peroxide, pectic acid, powdered agar-agar, sodium bicarbonate, sodium carbonate, sodium carboxymethyl starch or starch and other substances. The dissolution rate of a tablet or capsule may be also controlled by processing the contents into granulated forms, pellets, or other forms, by addition of binders, dissolution-control agents, or other excipients. The active substance may be contained in the capsule not only as a solid but also in solution or in suspension, e.g. in vegetable oil, polyethyleneglycol or glycerol, using surfactants, etc.

The pharmaceutical dosage form may include an enteric coating. As used herein, enteric coating refers to pharmaceutical controlled release methods to deliver a therapeutic dosage form to the gastrointestinal tract with a desired level of effective amount of active agents without the adverse gastrointestinal effects. Enteric coatings may be pH sensitive polymers designed to remain intact in the acidic environment of the stomach, but to dissolve in the more alkaline environment of the intestine. Enteric coatings may include by way of example, blends of cellulose acetate phthalate polymers (CAP), (see Wu et al, U.S. Pat. No. 5,356,634), cellulose acetate trimellitate polymers (CAT), (see Crook et al, U.S. Pat. No. 5,723,151), polyvinylpyrrolidone (PVP) with CAP and diethyl phthalate coating, (see Sipos, U.S. Pat. No. 4,079,125), polymers of an acrylic resin and an undercoat and overcoat of PVP, (see Patell, U.S. Pat. No. 4,775,536) and hydroxypropylmethyl cellulose phthalate (see Hodges et al, U.S. Pat No. 5,225,202). Other enteric coatings may include hydroxypropyl methylcellulose phthalate (HPMCP), hydroxypropyl methylcellulose acetate succinate (HPMCAS), polyvinyl acetate phthalate (PVAP) and acrylic resins. Preferably, disintegration of the enteric coating occurs in approximately 40 minutes, to correspond with the approximate time the therapeutic dosage form enters or is in the intestine.

As used herein, the terms “acceptable carrier” or “pharmaceutically acceptable carrier” are used interchangeably and refer to tablets, capsules, solvents, dispersion mediums, coatings, enteric coatings or delivery vehicles which may be used to administer the therapeutic dosage forms described herein without undue adverse physiological effects.

The present embodiments relate to pharmaceutical dosage forms containing as active agents a divalent cationic source of strontium, para-aminobenzoic acid other therapeutically effective compounds. Further additional optional ingredients may be added as described above.

The pharmaceutical dosage form may contain from about 250 to about 2000 milligrams of total active agents, or from about 500 to about 1500 milligrams of total active agents, or from about 500 to about 1250 milligrams of total active agents. The pharmaceutical dosage form may be administered orally, rectally or parenterally at a dose 500 to about 2000 milligrams of total active agents per day, or from about 500 to about 1500 milligrams of total active agents per day, or from about 500 to about 1250 milligrams of total active agents per day. The pharmaceutical dosage form may be administered in one or more dosage forms, for example, one or more tablets per day.

The effective dosage amount may be determined by routine experimentation, for example, by performing various pharmacological studies with mammalian models.

In one embodiment, a therapeutic dosage form for treating an arthritic condition is provided. The therapeutic dosage form comprises a divalent cationic source of strontium in an amount of from about 50 milligrams to about 400 milligrams, para-aminobenzoic acid and α-lipoic acid.

The therapeutic dosage form may comprise α-lipoic acid in amount of from about 50 milligrams to about 300 milligrams.

The therapeutic dosage form may comprises a divalent cationic source of strontium in an amount of from about 50 milligrams to about 400 milligrams, para-aminobenzoic acid in amount of from about 500 milligrams to about 2000 milligrams and α-lipoic acid in an amount of from about 50 milligrams to about 300 milligrams.

The therapeutic dosage form may comprises a divalent cationic source of strontium in an amount of from about 50 milligrams to about 400 milligrams, para-aminobenzoic acid in an amount of about 1000 milligrams and α-lipoic acid in an amount of about 150 milligrams.

In one embodiment, the therapeutic dosage form comprises a divalent cationic source of strontium in an amount of from about 50 milligrams to about 400 milligrams, para-aminobenzoic acid, α-lipoic acid and vitamin E.

The therapeutic dosage form may comprise vitamin E in an amount of from about 50 I.U. to about 300 I.U.

The therapeutic dosage form may comprise a divalent cationic source of strontium in an amount of from about 50 milligrams to about 400 milligrams, para-aminobenzoic acid in an amount of from about 500 milligrams to about 2000 milligrams, α-lipoic acid in an amount of from about 50 milligrams to about 300 milligrams and vitamin E in an amount of from about 50 I.U. to about 300 I.U.

The therapeutic dosage form may comprise a divalent cationic source of strontium in an amount of from about 50 milligrams to about 400 milligrams, para-aminobenzoic acid in an amount of about 1000 milligrams, α-lipoic acid in an amount of about 150 milligrams and vitamin E in an amount of about 200 I.U.

By way of example, a tablet may be provided comprising a therapeutically effective amount of a divalent cationic source of strontium, a para-aminobenzoic acid and α-lipoic acid. Thus, a single tablet may comprise a therapeutically effective amount of a divalent cationic source of strontium in an amount of from about 25 milligrams to about 200 milligrams, para-aminobenzoic acid in the amount from about 250 milligrams to about 1000 milligrams and α-lipoic acid in an amount of from about 25 milligrams to about 150 milligrams.

In an exemplary embodiment, a single tablet maybe provided comprising a therapeutically effective amount of a divalent cationic source of strontium in an amount of from about 25 milligrams to about 200 milligrams, a therapeutically effective amount of para-aminobenzoic acid in an amount of 500 milligrams and a therapeutically effective amount of α-lipoic acid in an amount of about 75 milligrams suitable for a daily regimen of two tablets per day.

In another example, a tablet may be provided comprising a therapeutically effective amount of a divalent cationic source of strontium, a para-aminobenzoic acid, α-lipoic acid and vitamin E. Thus, for example, a single tablet may be provided comprising a therapeutically effective amount of a divalent cationic source of strontium in an amount of from about 25 milligrams to about 200 milligrams, a therapeutically effective amount of para-aminobenzoic acid in an amount of from about 250 milligrams to about 1000 milligrams, a therapeutically effective amount of α-lipoic acid in an amount of from about 25 milligrams to about 150 milligrams and a therapeutically effective amount of vitamin E in an amount of from about 25 I.U. to about 150 I.U.

In an exemplary embodiment, a single tablet may be provided comprising a therapeutically effective amount of a divalent cationic source of strontium in an amount of from about 50 milligrams to about 400 milligrams, a therapeutically effective amount of para-aminobenzoic acid in an amount of 1000 milligrams and a therapeutically effective amount of α-lipoic acid in an amount of about 150 milligrams and a therapeutically effective amount of vitamin E in an amount of about 100 I.U. suitable for a daily regimen of two tablets per day.

In one embodiment, a method may be provided for treating symptoms or etiology of an arthritic condition in a human. The method comprises administering to a human in need thereof a therapeutically effective amount of a therapeutic dosage form, the therapeutic dosage form comprising a divalent cationic source of strontium, para-aminobenzoic acid, and α-lipoic acid.

The method may comprise administration of a therapeutically effective amount of a source of strontium in the amount of about 50 milligrams to about 400 milligrams.

The method may comprise administration of a therapeutically effective amount of para-aminobenzoic acid or salts thereof in the amount of about 500 milligrams to about 2000 milligrams.

The method may comprise administration of a therapeutically effective amount of α-lipoic acid in an amount of from about 50 milligrams to about 300 milligrams.

In another embodiment, a method may be provided for treating symptoms or etiology of an arthritic condition in a human. The method comprises administering to a human in need thereof a therapeutically effective amount of a therapeutic dosage form, the therapeutic dosage form comprising a divalent cationic source of strontium, para-aminobenzoic acid, α-lipoic acid, and vitamin E.

The method may comprise administering vitamin E in an amount of from about 50 I.U. to about 300 I.U.

The method may comprise administering a divalent cationic source of strontium selected from the group consisting of: strontium acetyl salicylate, strontium acetyloxy-benzoate, strontium ascorbate, strontium aspartate in either L and/or D-form, strontium benzenesulfonate, strontium clodronate, strontium carbonate, strontium citrate, strontium fumarate, strontium glutamate in either L- and/or D-form, strontium glutarate, strontium ibandronate, strontium ibuprofenate, strontium ketoprofenate, strontium maleate, strontium malonate, strontium methanesulfonate, strontium naproxenate, strontium oxalate, strontium pyruvate, strontium ranelate, strontium risedronate, strontium salicylate, strontium succinate, strontium tartrate, strontium L-threonate, strontium ranelate and mixtures thereof.

In one embodiment, the method may be directed to the treatment of symptoms or etiology of an arthritic condition such as osteoarthritis. The method may comprise administering to a human in need thereof one or more tablet comprising a therapeutically effective amount of a divalent cationic source of strontium, para-aminobenzoic acid and α-lipoic acid.

In one embodiment, the method may be directed to the treatment of symptoms or etiology of an arthritic condition such as rheumatoid arthritis. The method may comprise administering to a human in need thereof one or more tablet comprising a therapeutically effective amount of a divalent cationic source of strontium, a therapeutically effective amount of para-aminobenzoic acid, a therapeutically effective amount of α-lipoic acid and a therapeutically effective amount of vitamin E.

The divalent metal salts of the present embodiments, α-lipoic acid and vitamin E may have advantages over compositions without α-lipoic acid and vitamin E, e.g., providing potentially improved bioavailability and/or simultaneous treatment of bone/joint aliments, and/or symptoms and etiology of arthritic conditions, which may make it possible to administer reduced dosages of therapeutics in the treatment of arthritic conditions and/or simultaneously treat arthritic conditions and osteoporosis.

The methods herein described may provide treatment of the symptoms and etiology of an arthritic condition including, but not limited thereto, osteoarthritic condition, rheumatoid arthritic condition, juvenile chronic arthritis associated condition, juvenile idiopathic arthritis associated condition, Spondyloarthropathies, ankylosing spondylitis, Reiter's syndrome associated condition, condition associated with psoriatic arthritis, gout condition, condition associated with pseudogout (pyrophosphate arthritis), condition associated with systemic lupus erythematosus (SLE), condition associated with systemic sclerosis (scleroderma), condition associated with Behcet's disease, condition associated with relapsing polychondritis, condition associated with adult Still's disease, condition associated with transient regional osteoporosis, condition associated with neuropathic arthropathy, condition associated with sarcoidosis, arthritic condition, rheumatic condition, joint condition, osteoarthritis joint condition, rheumatoid arthritic joint condition, juvenile chronic arthritis associated joint condition, juvenile idiopathic arthritis associated joint condition, Spondyloarthropathic conditions, ankylosing spondylitis conditions, reactive arthritis (Reiter's syndrome) associated joint condition, joint condition associated with psoriatic arthritis, gout joint condition, joint condition associated with pseudogout (pyrophosphate arthritis), joint condition associated with systemic lupus erythematosus (SLE), joint condition associated with systemic sclerosis (scleroderma), joint condition associated with Behcet's disease, joint condition associated with relapsing polychondritis, joint condition associated with adult Still's disease, joint condition associated with transient regional osteoporosis, joint condition associated with neuropathic arthropathy, joint condition associated with sarcoidosis, arthritic joint condition, rheumatic joint condition, acute condition, acute joint condition, chronic condition, chronic joint condition, inflammatory condition, inflammatory joint condition, mechanical condition, mechanical joint condition, condition associated with the fibromyalgia syndrome (EMS), condition associated with polymyalgia rheumatics, monarticular joint condition and polyarticular joint condition.

In addition to strontium compounds, para-aminobenzoic acid and α-lipoic acid, and strontium compounds, para-aminobenzoic acid α-lipoic acid and vitamin E, the method of treating symptoms or etiology of an arthritic condition in a subject in need thereof may further include in the therapeutic dosage form one or more therapeutically and/or prophylactically active substances.

In one embodiment, a pharmaceutical dosage form may be provided comprising a divalent cationic source of strontium in the amount of about 50 milligrams to about 400 milligrams, para-aminobenzoic acid, vitamin B₆, vitamin B₁₂ and folic acid or folate.

The pharmaceutical dosage form may comprise a divalent cationic source of strontium in the amount of about 50 milligrams to about 400 milligrams, para-aminobenzoic acid in the amount of about 500 milligrams to about 2000 milligrams, vitamin B₆ in the amount of about 10 milligrams to about 50 milligrams, vitamin B₁₂ in the amount of about 1 milligrams to about 3 milligrams and folic acid or folate in the amount of about 0.5 milligrams to about 3 milligrams.

The pharmaceutical dosage form may comprise a divalent cationic source of strontium in the amount of about 50 milligrams to about 400 milligrams, para-aminobenzoic acid in the amount of about 1000 milligrams, vitamin B₆ in the amount of about 25 milligrams, vitamin B₁₂ in the amount of about 2 milligrams and folic acid or folate in the amount of about 1.5 milligrams. It may be advantageous to distribute the total dosage of a divalent cationic source of strontium, PABA, vitamin B₆, vitamin B₁₂ and folic acid or folate among more than one pharmaceutical dosage forms, for example, two or more tablets to be administered per day.

For example, a single tablet may be provided comprising a therapeutically effective amount of a strontium source, para-aminobenzoic acid, vitamin B₆, vitamin B₁₂, folic acid or folate suitable for a two tablet per day regimen. In each tablet, the divalent cationic source of strontium may be present in the amount of about 25 milligrams to about 200 milligrams, para-aminobenzoic acid may be present in the amount of from about 250 milligrams to about 750 milligrams, vitamin B₆ may be present in the amount of from about 7.5 milligrams to about 25 milligrams, vitamin B₁₂ may be present in the amount of from about 0.5 milligrams to about 1.5 milligrams, and folic acid or folate may be present in the amount of from about 0.25 milligrams to about 1.5 milligrams.

In an exemplary embodiment, the single tablet may comprise a divalent cationic source of strontium in the amount of about 25 milligrams to about 200 milligrams, para-aminobenzoic acid in the amount of about 500 milligrams, vitamin B₆ in the amount of about 12.5 milligrams, vitamin B₁₂ in the amount of about 1 milligrams, and folic acid or folate is present in the amount of about 0.75 milligrams, suitable for a two tablet per day regimen.

In one embodiment, a method may be provided for treating symptoms or etiology of osteoporosis in a subject. The method comprises the step of administering to a mammal in need thereof a therapeutically effective amount of a pharmaceutical dosage form comprising a divalent cationic source of strontium and a combination of para-aminobenzoic acid, vitamin B₆, vitamin B₁₂ and folic acid or folate.

The method may comprise the step of administering a pharmaceutical dosage form comprising a divalent cationic source of strontium in the amount of about 50 milligrams to about 400 milligrams, para-aminobenzoic acid in the amount of about 500 milligrams to about 2000 milligrams, vitamin B₆ in the amount of from about 10 milligrams to about 50 milligrams, vitamin B₁₂ in the amount of from about 1 milligrams to about 3 milligrams and folic acid or folate in the amount of from about 0.5 milligrams to about 3 milligrams.

In an exemplary embodiment, a divalent source of strontium in the amount of about 100 milligrams to about 400 milligrams, para-aminobenzoic acid in the amount of about 1000 milligrams, vitamin B₆ is in the amount of about 25 milligrams, vitamin B₁₂ in the amount of about 2 milligrams, and folic acid or folate in the amount of about 1.5 milligrams may be administered as a daily regimen. In another exemplary embodiment, the method comprises administering a divalent cationic source of strontium in the amount of about 25 milligrams to about 200 per tablet, PABA in the amount of about 500 milligrams per tablet, vitamin B₆ in the amount of about 12.5 milligrams per tablet, B₁₂ in the amount of about 1 milligrams per tablet, folic acid in the amount of about 0.75 milligrams per tablet, administered as a two tablet per day regimen.

Tablets may contain one or more excipients or vehicles chosen from diluents, lubricants, binders, disintegrating agents, absorbents, colorants, sweeteners and the like. By way of example and without limitation the diluents may include lactose, dextrose, sucrose, mannitol, sorbitol, cellulose, and/or glycerin. The lubricants may include silica, talc, stearic acid and its magnesium and calcium salts, and/or polyethyleneglycol. The binders may include aluminum and magnesium silicate, starch, gelatin, tragacanth, methyl-cellulose, sodium carboxymethylcellulose and/or polyvinylpyrrolidone. The disintegrants may include agar, alginic acid and its sodium salt and/or effervescent mixtures.

The tablet may further comprise an enteric coating such that the therapeutic dosage form is controllably released into the gastrointestinal tract when administered orally.

Utilization of a divalent cationic source of strontium and para-aminobenzoic acid, vitamin B₆, vitamin B₁₂ and folic acid or folate as described hereinabove, may result in increase of bone mass, and/or reduction of pain and/or increase of joint mobility for most individuals in need thereof.

In addition to strontium compounds, PABA vitamin B₆, vitamin B₁₂ and folic acid or folate, the method of treating symptoms or etiology of osteoporosis in a subject in need thereof may further include in the therapeutic dosage form one or more therapeutically and/or prophylactically active substances.

The divalent cationic source of strontium of the present embodiments, para-aminobenzoic acid, vitamin B₆, vitamin B₁₂ and folic acid or folate may have advantages over strontium compositions alone. For example, potentially improved bioavailability and simultaneous treatment of bone/joint aliments or degeneration may make it possible to administer reduced dosages of therapeutics in the treatment of osteoporosis and/or arthritic conditions, particularly when both osteoporosis and arthritic conditions are present in a subject.

Para-aminobenzoic acid (PABA) is generally believed to be an anti-oxidant/anti-free radical agent. It is also believed that aldehyde chemical metabolites containing carbonyl functional groups may be generated during the process of chronic inflammation. These aldehyde products may result from the degradation of unsaturated fatty acids in the course of pathologically increased lipid peroxidation, which may be initiated by a variety of activated oxygen chemical species such as hydroxyl radicals. This process is sometimes referred to as the nonenzymatic inflammatory cascade. The reactive cascade of free radical propagation is believed to include lipid peroxidation followed by aldehyde formation and other subsequent effects of inflammation. Aldehyde products of this reactive cascade are believed to react with free amino groups of proteins, nucleic acids and phospholipids to form Schiff bases and/or to function as chemotactic agents, which may attract white blood cells to sites of such inflammation. The activated oxygen chemical species released by white blood cells may further exacerbate the inflammatory process. PABA, a primary amine derivative of benzoic acid, may function therapeutically by chemically binding to and sequestering the aldehyde and/or ketone products of lipid peroxidation. Increased levels of lipid peroxidation may contribute, in part, to the non-enzymatic inflammatory cascade process which may cause the secondary etiology of chronic inflammatory diseases, such as rheumatoid arthritis.

The exact mechanism of biological action which may account for increased bone mass or reduction of bone mass loss and/or alleviation of arthritic induced pain and inflammation when a divalent cationic source of strontium is used, vitamim B actives and optionally PABA is not known. However, this combination is believed to contribute to the prevention of bone mass loss, cellular regeneration, muscular tissue maintenance and tissue recovery acceleration and thus account for its potential beneficial effects in controlling and/or preventing osteoporosis, osteoarthritic and arthritic inflammation when administered as herein disclosed. Increased bone mass or reduction of bone mass loss by the therapeutic dosage form described herein may include, for example, generating new or additional bone at locations where such bone growth is not presently taking place and/or stimulating the growth of bone which is already in the process of formation. Increased bone mass or reduction of bone mass loss may take place due to the combined effects of the divalent source of strontium and vitamin B₆, vitamin B₁₂ and folic acid or folate and/or PABA by increasing osteoblast activity in the subject and may further be coupled with an elevation at least one bone anabolic agent in the subject. An example of a bone anabolic agent endogenously produced in the human body may be parathyroid hormone (PTH). The effects of PABA on alleviating or eliminating possible inflammation in combination with increased bone mass or reduction of bone mass loss of the divalent source of strontium and vitamin B₆, vitamin B₁₂ and folic acid or folate may provide a more desirable and/or effective regimen, for example, for subjects suffering from both rheumatoid arthritis and osteoporosis.

Other optional vitamins and mineral components may be included in the therapeutic dosage form and methods disclosed herein. These additional components may include iodine, calcium, potassium, iron, magnesium, manganese, zinc and selenium, preferably in the form of chelates, vitamins A, B, D, choline bitartrate, inositol, pantothenic acid, nicotinic acid, biotin, rutin, betain and glutamic acid.

The therapeutic dosage form may further include one or more therapeutically and/or prophylactically active substances. Such active substances may include agents effective in the treatment of or acting on joint tissue components or bone or for increasing bone mass or reducing bone mass loss. For example, and without limitation, such agents may include anabolic agents, analgesic agents, antiresorptive agents aromatase inhibitors, chondroitin sulphate, COX-2 inhibitors, COX-3 inhibitors, disease modifying anti-rheumatic compounds (DMARDs), glucocorticoids, glucosamine, glycine antagonists, inhibitors of inducible nitric oxide synthetase (iNOS), inhibitors of interleukin-1 converting enzyme, inhibitors of matrix metallo-proteinases (MMPs), inhibitors/antagonists of IL-1, inhibitors/antagonists of RANK-ligand, inhibitors/antagonists of TNF-oc, N-acetylcholine receptor agonists, neurokinin antagonists, neuroleptic agents, NMDA receptor antagonists, non-steroidal anti-inflammatory agents (NSAIDs), opioids, pallitative agents, PAR2 receptor antagonists, selective estrogen receptor modulators (SERMs) and vanilloid receptor antagonists. Additional therapeutically and/or prophylactically active substances may include adjuvants, anti-infective agents, anti-inflammatory agents, antioxidants, glycosaminoglycans, herbal derivatives and mixtures thereof. Anabolic agents may include natural and truncated forms of parathyroid hormone (PTH) including aminated natural and truncated forms thereof, anabolic Vitamin D analogs, a low-density lipoprotein receptor-related protein 5, an activator of non-genomic estrogen-like signaling, a bone morphogenic protein (BMP), an insulin-like growth factor (IGF), a fibroblast growth factor (FGF), sclerostin, leptin, a prostaglandin, a statin, a growth hormone, a growth hormone releasing factor (GHRF), hepatocyte growth factor (HGF), calcitonin gene related peptide (CGRP), parathyroid hormone related peptide (PTHrP), transforming growth factor (TGF)-β1 and combinations thereof. Antiresorptive agents may include human calcitonin, non-human calcitonin, calcitonin gene related peptide (CGRP), hormone replacement therapy (HRT) agents such as selective estrogen receptor modulators, bisphosphonates, cathepsin-K inhibitors, and various combinations thereof.

### EXAMPLES

The following examples are illustrative of the embodiments of the present invention and are not to be interpreted as limiting or restrictive. Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements (e.g., weights). Thus, in one example, a tablet composition may be prepared by combining the following: 350 milligrams of strontium gluconate, 500 milligrams of PABA, and 75 milligrams of α-lipoic acid along with acceptable excipients, and processed into tablets. The tablets may further include enteric coatings. A daily regimen of two tablets as just described may provide effective treatment to a subject with an arthritic condition, such as osteoarthritis, or a subject with osteoarthritis and an osteoporitic condition.

In one example, a tablet suitable for a two tablet per day regimen may be prepared by combining the following: about 350 milligrams of strontium gluconate, about 500 milligrams of PABA, and about 75 milligrams of α-lipoic acid along with acceptable excipients, and processed into tablets. The tablets may further include enteric coatings. A daily regimen of two tablets as just described may provide effective treatment to a subject with an arthritic condition, such as rheumatoid arthritis, or a subject with rheumatoid arthritis and an osteoporitic condition.

In another example, a tablet suitable for a two tablet per day regimen may be prepared by combining the following: about 350 milligrams of strontium gluconate, about 500 milligrams of PABA, about 75 milligrams of α-lipoic acid, and about 100 I.U. vitamin E along with acceptable excipients, and processed into tablets. The tablets may further include enteric coatings. A daily regimen of two tablets as just described may provide effective treatment to a subject with an arthritic condition, such as rheumatoid arthritis, or a subject with rheumatoid arthritis and an osteoporitic condition.

In one example, a tablet suitable for a two tablet per day regimen may be prepared by combining the following: about 325 milligrams of strontium gluconate (approximately 60 milligrams of divalent cationic strontium), about 500 milligrams of PABA, about 12.5 milligrams of vitamin B₆, about 1 milligrams of vitamin B₁₂, and about 0.75 milligrams of folic acid, along with acceptable excipients, and processed into tablets. The tablets may further include enteric coatings. A daily regimen of two tablets as just described may provide effective treatment to a subject with an osteoporitic condition or a subject with an osteoporitic condition and an arthritic condition, such as rheumatoid arthritis.

As used herein, “comprising,” “including,” “containing,“ “characterized by,” and grammatical equivalents thereof are inclusive or open-ended terms that do not exclude additional, unrecited elements or method steps. “Comprising” is to be interpreted as including the more restrictive terms “consisting of” and “consisting essentially of.”

As used herein, “consisting of” and grammatical equivalents thereof exclude any element, step, or ingredient not specified in the claim.

As used herein, “consisting essentially of” and grammatical equivalents thereof limit the scope of a claim to the specified materials or steps and those that do not materially affect the basic and novel characteristic or characteristics of the claimed invention.

While the invention has been described in detail and with reference to specific embodiments thereof, it will be apparent to one skilled in the art that various changes and modifications can be made without departing from the spirit and scope of the invention.

What is claimed is:

1. A therapeutic dosage form comprising a divalent cationic source of strontium in an amount from about 50 milligrams to about 400 milligrams, para-aminobenzoic acid and α-lipoic acid.

2. The therapeutic dosage form of claim 1, wherein the para-aminobenzoic acid is in an amount of from about 500 milligrams to about 2000 milligrams.

3. The therapeutic dosage form of claim 1, wherein the α-lipoic acid is in an amount of from about 50 milligrams to about 300 milligrams.

4. The therapeutic dosage form of claim 1, wherein the divalent cationic source of strontium is in an amount of from about 50 milligrams to about 400 milligrams, para-aminobenzoic acid is in an amount of from about 500 milligrams to about 2000 milligrams and α-lipoic acid is in an amount of from about 50 milligrams to about 300 milligrams.

5. The therapeutic dosage form of claim 1, wherein the divalent cationic source of strontium is in an amount of from about 50 milligrams to about 400 milligrams, para-aminobenzoic acid is in an amount of about 1000 milligrams and α-lipoic acid is in an amount of about 150 milligrams.

6. The therapeutic dosage form of claim 1, further comprising vitamin E.

7. The therapeutic dosage form of claim 6, wherein vitamin E is in an amount of from about 50 I.U. to about 300 I.U.

8. The therapeutic dosage form of claim 6, wherein the divalent cationic source of strontium in an amount of from about 50 milligrams to about 400 milligrams, para-aminobenzoic acid is in an amount of from about 500 milligrams to about 2000 milligrams, α-lipoic acid is in an amount of from about 50 milligrams to about 300 milligrams and vitamin E is in an amount of from about 50 I.U. to about 300 I.U.

9. The therapeutic dosage form of claim 6, wherein the divalent cationic source of strontium in an amount of from about 50 milligrams to about 400 milligrams, para-aminobenzoic acid is in an amount of about 1000 milligrams, α-lipoic acid is in an amount of about 150 milligrams and vitamin E is in an amount of from about 200 I.U.

10. The therapeutic dosage form of claim 1, wherein the divalent cationic source of strontium is selected from the group consisting of: strontium acetyl salicylate, strontium acetyloxy-benzoate, strontium ascorbate, strontium aspartate in either L and/or D-form, strontium benzenesulfonate, strontium clodronate, strontium carbonate, strontium citrate, strontium fumarate, strontium glutamate in either L- and/or D-form, strontium glutarate, strontium ibandronate, strontium ibuprofenate, strontium ketoprofenate, strontium maleate, strontium malonate, strontium methanesulfonate, strontium naproxenate, strontium oxalate, strontium pyruvate, strontium ranelate, strontium risedronate, strontium salicylate, strontium succinate, strontium tartrate, strontium L-threonate, strontium ranelate and mixtures thereof.

11. The therapeutic dosage form of claim 1, further comprises an acceptable carrier suitable for oral administration.

12. The therapeutic dosage form of claim 1, further comprises an enteric coating such that the therapeutically effective composition is controllably released into the gastrointestinal tract when administered orally.

13. A method for treating symptoms or etiology of an arthritic condition in a human comprising administering to a human in need thereof a therapeutically effective amount of a therapeutic dosage form, the therapeutic dosage form comprising a divalent cationic source of strontium, para-aminobenzoic acid and α-lipoic acid.

14. The method of claim 13, wherein the source of strontium is about from about 50 milligrams to about 400 milligrams.

15. The method of claim 13, wherein the para-aminobenzoic acid is from about 500 milligrams to about 2000 milligrams.

16. The method of claim 13, wherein the α-lipoic acid is from about 50 milligrams to about 300 milligrams.

17. The method of claim 13, further comprising vitamin E.

18. The method of claim 17, wherein vitamin E is in an amount of from about 50 I.U. to about 300 I.U.

19. The method of claim 13, wherein the divalent cationic source of strontium is selected from the group consisting of: strontium acetyl salicylate, strontium acetyloxy-benzoate, strontium ascorbate, strontium aspartate in either L and/or D-form, strontium benzenesulfonate, strontium clodronate, strontium carbonate, strontium citrate, strontium fumarate, strontium glutamate in either L- and/or D-form; strontium glutarate, strontium ibandronate, strontium ibuprofenate, strontium ketoprofenate, strontium maleate, strontium malonate, strontium methanesulfonate, strontium naproxenate, strontium oxalate, strontium pyruvate, strontium ranelate, strontium risedronate, strontium salicylate, strontium succinate, strontium tartrate, strontium L-threonate, strontium ranelate and mixtures thereof.

20. The method of claim 13, wherein the arthritic condition is selected from the group consisting of an osteoarthritic condition, rheumatoid arthritic condition, juvenile chronic arthritis associated condition, juvenile idiopathic arthritis associated condition, Spondyloarthropathies, ankylosing spondylitis, Reiter's syndrome associated condition, condition associated with psoriatic arthritis, gout condition, condition associated with pseudogout (pyrophosphate arthritis), condition associated with systemic lupus erythematosus (SLE), condition associated with systemic sclerosis (scleroderma), condition associated with Behcet's disease, condition associated with relapsing polychondritis, condition associated with adult Still's disease, condition associated with transient regional osteoporosis, condition associated with neuropathic arthropathy, condition associated with sarcoidosis, arthritic condition, rheumatic condition, joint condition, osteoarthritis joint condition, rheumatoid arthritic joint condition, juvenile chronic arthritis associated joint condition, juvenile idiopathic arthritis associated joint condition, Spondyloarthropathic conditions, ankylosing spondylitis conditions, reactive arthritis (Reiter's syndrome) associated joint condition, joint condition associated with psoriatic arthritis, gout joint condition, joint condition associated with pseudogout (pyrophosphate arthritis), joint condition associated with systemic lupus erythematosus (SLE), joint condition associated with systemic sclerosis (scleroderma), joint condition associated with Behcet's disease, joint condition associated with relapsing polychondritis, joint condition associated with adult Still's disease, joint condition associated with transient regional osteoporosis, joint condition associated with neuropathic arthropathy, joint condition associated with sarcoidosis, arthritic joint condition, rheumatic joint condition, acute condition, acute joint condition, chronic condition, chronic joint condition, inflammatory condition, inflammatory joint condition, mechanical condition, mechanical joint condition, condition associated with the fibromyalgia syndrome (EMS), condition associated with polymyalgia rheumatics, monarticular joint condition and polyarticular joint condition.

21. The method of claim 13, further comprising administering a therapeutically effective amount of one or more members selected from the group consisting of anabolic agents, analgesic agents, antiresorptive agents aromatase inhibitors, chondroitin sulphate, COX-2 inhibitors, COX-3 inhibitors, disease modifying anti-rheumatic compounds (DMARDs), glucocorticoids, glucosamine, glycine antagonists, inhibitors of inducible nitric oxide synthetase (iNOS), inhibitors of interleukin-1 converting enzyme, inhibitors of matrix metallo-proteinases (MMPs), inhibitors/antagonists of IL-1, inhibitors/antagonists of RANK-ligand, inhibitors/antagonists of TNF-oc, N-acetylcholine receptor agonists, neurokinin antagonists, neuroleptic agents, NMDA receptor antagonists, non- steroidal anti-inflammatory agents (NSAIDs), opioids, pallitative agents, PAR2 receptor antagonists, selective estrogen receptor modulators (SERMs), vanilloid receptor antagonists, adjuvants, alpha-lipoic acid, anti-infective agents, anti-inflammatory agents, antioxidants, glycosaminoglycans, herbal derivatives, natural and truncated forms of parathyroid hormone (PTH), aminated natural and truncated forms of parathyroid hormone (PTH), anabolic Vitamin D analogs, low-density lipoprotein receptor-related protein 5, non-genomic estrogen-like signaling activator, bone morphogenic protein (BMP), insulin-like growth factor (IGF), fibroblast growth factor (FGF), sclerostin, leptin, a prostaglandin, statin, growth hormone, growth hormone releasing factor (GHRF), hepatocyte growth factor (HGF), calcitonin gene related peptide (CGRP), parathyroid hormone related peptide (PTHrP), transforming growth factor (TGF)-β1, human calcitonin, non-human calcitonin, calcitonin gene related peptide (CGRP), hormone replacement therapy (HRT) agents, selective estrogen receptor modulator, bisphosphonates, and cathepsin-K inhibitors.

22. A therapeutic dosage form comprising a divalent cationic source of strontium of at least about 50 milligrams and a combination of para-aminobenzoic acid, vitamin B₆, vitamin B₁₂ and folic acid or folate.

23. The therapeutic dosage form of claim 22, wherein the divalent cationic source of strontium is from about 50 milligrams to about 400 milligrams, the para-aminobenzoic acid is from about 500 milligrams to about 2000 milligrams, vitamin B₆ is from about 10 milligrams to about 50 milligrams, vitamin B₁₂ is from about 1 milligrams to about 3 milligrams and folic acid or folate is from about 0.5 milligrams to about 3 milligrams.

24. The therapeutic dosage form of claim 22, wherein the a divalent cationic source of strontium is from about 50 milligrams to about 400 milligrams, para-aminobenzoic acid is about 1000 milligrams, vitamin B₆ is about 25 milligrams, vitamin B₁₂ is about 2 milligrams, and folic acid or folate is about 1.5 milligrams.

25. The therapeutic dosage form of claim 22, wherein the source of divalent strontium is selected from the group consisting of: strontium acetyl salicylate, strontium acetyloxy-benzoate, strontium ascorbate, strontium aspartate in either L and/or D-form, strontium benzenesulfonate, strontium butyrate, strontium camphorate, strontium carbonate, strontium clodronate, strontium chloride, strontium citrate, strontium ethanesulfonate, strontium fumarate, strontium gluconate, strontium glutamate in either L- and/or D-form, strontium glutarate, strontium ibandronate, strontium ibuprofenate, strontium ketoprofenate, strontium lactate, strontium L-threonate, strontium malate, strontium maleate, strontium maleate, strontium malonate, strontium methanesulfonate, strontium naproxenate, strontium nitrate, strontium oxalate, strontium phosphate, strontium pyruvate, strontium ranelate, strontium risedronate, strontium salicylate, strontium succinate, strontium sulfate, strontium tartrate and mixtures thereof.

26. The therapeutic dosage form of claim 22, further comprises an acceptable carrier suitable for oral administration.

27. The therapeutic dosage form of claim 22, further comprises an enteric coating such that the therapeutically effective composition is controllably released into the gastrointestinal tract when administered orally.

28. A method for treating symptoms or etiology of osteoporosis in a subject comprising the step of administering to a mammal in need thereof a therapeutically effective amount of a therapeutic dosage form comprising a divalent cationic source of strontium, and a combination of para-aminobenzoic acid, vitamin B₆, vitamin B₁₂ and folic acid or folate.

29. The method of claim 28, wherein the a divalent cationic source of strontium is from about 50 milligrams to about 400 milligrams, para-aminobenzoic acid is from about 500 milligrams to about 2000 milligrams, vitamin B₆ is from about 10 milligrams to about 50 milligrams, vitamin B₁₂ is from about 1 milligrams to about 3 milligrams and folic acid or folate is from about 0.5 milligrams to about 3 milligrams.

30. The method of claim 28, wherein the divalent cationic source of strontium is from about 50 milligrams to about 400 milligrams, para-aminobenzoic acid is about 1000 milligrams, vitamin B₆ is from about 25 milligrams, vitamin B₁₂ is about 2 milligrams, and folic acid or folate is about 1.5 milligrams.

31. The method of claim 28, wherein the therapeutically effective amount of the divalent source of strontium is selected from the group consisting of: strontium acetyl salicylate, strontium acetyloxy-benzoate, strontium ascorbate, strontium aspartate in either L and/or D-form, strontium benzenesulfonate, strontium butyrate, strontium camphorate, strontium carbonate, strontium clodronate, strontium chloride, strontium citrate, strontium ethanesulfonate, strontium fumarate, strontium gluconate, strontium glutamate in either L- and/or D-form, strontium glutarate, strontium ibandronate, strontium ibuprofenate, strontium ketoprofenate, strontium lactate, strontium L-threonate, strontium malate, strontium maleate, strontium maleate, strontium malonate, strontium methanesulfonate, strontium naproxenate, strontium nitrate, strontium oxalate, strontium phosphate, strontium pyruvate, strontium ranelate, strontium risedronate, strontium salicylate, strontium succinate, strontium sulfate, strontium tartrate and mixtures thereof.

32. The method of claim 28, further comprising administering a therapeutically effective amount of one or more members selected from the group consisting of anabolic agents, analgesic agents, antiresorptive agents aromatase inhibitors, chondroitin sulphate, COX-2 inhibitors, COX-3 inhibitors, disease modifying anti-rheumatic compounds (DMARDs), glucocorticoids, glucosamine, glycine antagonists, inhibitors of inducible nitric oxide synthetase (iNOS), inhibitors of interleukin-1 converting enzyme, inhibitors of matrix metallo-proteinases (MMPs), inhibitors/antagonists of IL-1, inhibitors/antagonists of RANK-ligand, inhibitors/antagonists of TNF-oc, N-acetylcholine receptor agonists, neurokinin antagonists, neuroleptic agents, NMDA receptor antagonists, non-steroidal anti-inflammatory agents (NSAIDs), opioids, pallitative agents, PAR2 receptor antagonists, selective estrogen receptor modulators (SERMs), vanilloid receptor antagonists, adjuvants, alpha-lipoic acid, anti-infective agents, anti-inflammatory agents, antioxidants, glycosaminoglycans, herbal derivatives, natural and truncated forms of parathyroid hormone (PTH), aminated natural and truncated forms of parathyroid hormone (PTH), anabolic Vitamin D analogs, low-density lipoprotein receptor-related protein 5, non-genomic estrogen-like signaling activator, bone morphogenic protein (BMP), insulin-like growth factor (IGF), fibroblast growth factor (FGF), sclerostin, leptin, a prostaglandin, statin, growth hormone, growth hormone releasing factor (GHRF), hepatocyte growth factor (HGF), calcitonin gene related peptide (CGRP), parathyroid hormone related peptide (PTHrP), transforming growth factor (TGF)-β1, human calcitonin, non-human calcitonin, calcitonin gene related peptide (CGRP), hormone replacement therapy (HRT) agents, selective estrogen receptor modulator, bisphosphonates, and cathepsin-K inhibitors.
